(19) United States
(12) Patent Application Publication
(10) Pub. No.: US 2015/0018783 A1
(43) Pub. Date: Jan. 15, 2015

(54) SYSTEM, METHOD AND APPARATUS FOR SILENT SUCTION TUBING

(71) Applicants: Linda Louise MYERS, Clinton, PA (US); Earl Frederick Myers, Clinton, PA (US)

(72) Inventors: Linda Louise MYERS, Clinton, PA (US); Earl Frederick Myers, Clinton, PA (US)

(21) Appl. No.: 13/941,039

(22) Filed: Jul. 12, 2013

(51) Int. Cl. A61M 1/00; A61M 39/28

(52) U.S. Cl. CPC A61M 1/008; A61M 39/28; A61M 2039/0009
USPC 604/319

(57) ABSTRACT

An apparatus for silent suction tubing may be disclosed. A surgical tube clamp may have a hollow body with a first end, a second end, and a bottom surface between the first end and the second end. A vertical arm may extend from the first end and have an inner face with a plurality of teeth. A horizontal arm may extend from the second end toward the first end. The horizontal arm may have a beveled edge proximate the inner face of the vertical arm. A first protrusion on the bottom surface may extend within the hollow body and a second protrusion on the horizontal arm may extend within the hollow body.

### BACKGROUND

During most medical or surgical procedures, suctioning is used to remove excess fluids from an operative field. Suctioning is important to clearly expose the operative field to allow a surgeon or other medical professional to view the area and safely and effectively perform the procedure. Suction is usually created using any one of a number of mechanical or electrical devices such as battery or electrically controlled pumps. In use, these devices act to draw fluids into a tube or hose.

Generally, the suction should be consistently and immediately available to the surgeon or medical professional during the procedure. Therefore the suction device should remain on throughout. Unfortunately the constant running of the suction device creates a loud sound that medical personnel find annoying and distracting. In some circumstances, medical personnel will attempt to use other expensive surgical equipment to attempt to silence the suction noise, which can damage the equipment, or render the suction unavailable for immediate use.

### SUMMARY

According to at least one exemplary embodiment, an apparatus for silent suction tubing may be disclosed. A surgical tube clamp may have a hollow body with a first end, a second end, and a bottom surface between the first end and the second end. A vertical arm may extend from the first end and have an inner face with a plurality of teeth. A horizontal arm may extend from the second end toward the first end. The horizontal arm may have a beveled edge proximate the inner face of the vertical arm. A first protrusion on the bottom surface may extend within the hollow body and a second protrusion on the horizontal arm may extend within the hollow body.

In another exemplary embodiment, a system for silent surgical tubing may be disclosed. The system may include a surgical clamp with a hollow body having a first end, a second end, and a bottom surface between the first end and the second end. The first end may have a first aperture, and the second end may have a second aperture. A vertical arm may extend from the first end and have an inner face with a plurality of teeth. A horizontal arm may extend from the second end toward the first end. The horizontal arm may have a beveled edge proximate the inner face of the vertical arm. A first protrusion on the bottom surface may extend within the hollow body and a second protrusion on the horizontal arm may extend within the hollow body. A surgical tube may be received through the first aperture and the second aperture, and is operable to draw a fluid in a direction from the first end to the second end of the surgical clamp.

### BRIEF DESCRIPTION OF THE DRAWINGS

Advantages of embodiments of the present invention will be apparent from the following detailed description of the exemplary embodiments. The following detailed description should be considered in conjunction with the accompanying figures in which:

FIG. 1 is an exemplary embodiment of a surgical tube clamp.

FIG. 2a is a side view of an exemplary embodiment of a surgical tube clamp in an open position.

FIG. 2b is a side view of an exemplary embodiment of a surgical tube clamp in a partially closed position.

FIG. 2c is a side view of an exemplary embodiment of a surgical tube clamp in a closed position.

### DETAILED DESCRIPTION

Aspects of the present invention are disclosed in the following description and related figures directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

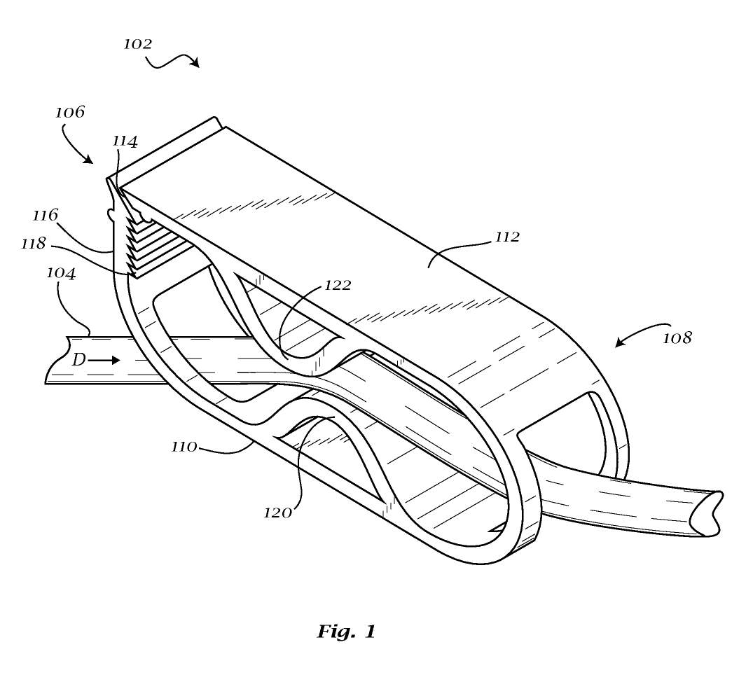

Turning now to FIG. 1, an exemplary embodiment of an apparatus for silent suction tubing may be shown. Silent suction tubing apparatus may include a clamp 102 and surgical tubing 104. Surgical tubing 104 may be, for example, sterile surgical tubing including surgical grade plastic tubing or similar material as would be understood by a person of ordinary skill in the art. Surgical tubing 104 may be, for example, coupled at one end to a suction device (not shown) and at the other end to a suction tip, and operable to draw fluid through the suction tip in a first direction D away from the operative field during an operation.

Clamp 102 may have a first end 106, a hollow portion 107 and a second end 108. Surgical tubing 104 may be received through first end 106, hollow portion 107, and second end 108 such that first end 106 is upstream from a suction direction of second end 108. In one exemplary embodiment, first end 106 and second end 108 may have complimentary apertures to allow surgical tubing to be received through clamp 102. The apertures may be square, rectangular, circular, or any shape that may allow passage of surgical tubing as would be understood by a person of ordinary skill in the art.

In some exemplary embodiments, clamp 102 may be operable to slide along a length of surgical tubing 104. In other exemplary embodiments, connecting end pieces may be added to surgical tubing 104 on either side of clamp 102, which may eliminate the possibility of clamp 102 from coming off of the surgical tubing 104 and becoming lost in the operative field.

Clamp 102 may have a bottom surface 110 between respective bottom portions of first end 106 and second end 108. In some exemplary embodiments, first end 106 and second end 108 may have substantially rounded edges, and bottom surface 110 may be substantially elongated and flat such that clamp 102 may have a generally oval or oblong shape between first end 106 and second end 108.

A horizontal arm 112 may extend from a top portion of second end 108 toward first end 106. In some exemplary embodiments horizontal arm 112 may be flexible but resilient with respect to second end 108. Horizontal arm 112 may have a distal end with beveled edge 114.

A vertical arm 116 may extend from a top portion of first end 106. Vertical arm 116 may be substantially perpendicular to horizontal arm 112. Vertical arm 116 may have a plurality of teeth 118. Plurality of teeth 118 may be, for example, triangular teeth that slope in a downward direction. In alternative embodiments, plurality of teeth 118 may be any contemplated shape or size without deviating from the scope of the present invention. Plurality of teeth 118 may be on a distal end and on an inner face of vertical arm 116. Plurality of teeth 118 may be proximate the beveled edge 114 of horizontal arm 112.

Bottom surface 110 may have a first protrusion 120 between first end 106 and second end 108 that may extend substantially toward and within hollow portion 107. Horizontal arm 112 may have a second protrusion 122 between beveled edge 114 and second end 108 that may extend substantially toward and within hollow portion 107. In one exemplary embodiment, first protrusion 120 and second protrusion 122 may be substantially rounded protrusions. In alternative embodiments, first protrusion 120 and second protrusion 122 may be any contemplated size or shape without deviating from the scope of the present invention.

First protrusion 120 may be substantially opposite second protrusion 122. For example, first protrusion 120 and second protrusion 122 may be situated on opposite sides of surgical tubing 104. In one exemplary embodiment, first protrusion 120 and second protrusion 122 may be in contact with surgical tubing 104. As explained in more detail below, horizontal arm 112 and vertical arm 116 may be manipulated such that a distance between first protrusion 120 and second protrusion 122 is modified. For example, the distance between first protrusion 120 and second protrusion 122 may be modified to increase or decrease a force, for example, a pinching or compressing force, exerted on surgical tubing 104.

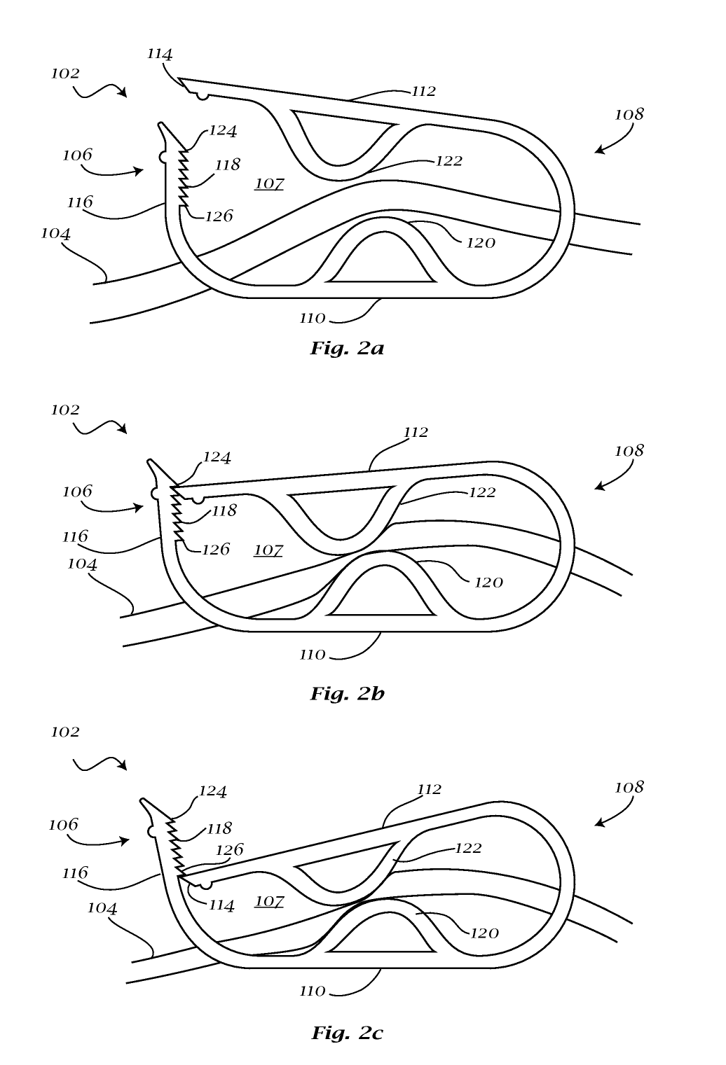

Referring now to FIGS. 2a-c, clamp 102 may be shown in a variety of exemplary positions. For example, FIG. 2a may show clamp 100 in an open position. In the open position, beveled edge 114 of horizontal arm 112 is not in contact with any of the plurality of teeth 118 of vertical arm 116. In this exemplary embodiment, first protrusion 120 and second protrusion 122 may be a maximum distance apart, and may exert a minimal, if any, force on surgical tubing 104. Thus, in the open position of FIG. 2a, suction through surgical tubing 104 may be substantially unimpeded. Further, clamp 102 may be free to slide along a length of surgical tubing 104.

FIG. 2b may show clamp 102 in a first closed position. For example, in the first closed position, beveled edge 114 of horizontal arm 112 may engage a first tooth 124 of said plurality of teeth 118 of vertical arm 116. In this exemplary embodiment, beveled edge 114 may act, for example as a pawl, and plurality of teeth 118 may act, for example, as a rack, as would be understood by a person of ordinary skill in the art. As such, the interaction of beveled edge 114 and plurality of teeth 118 may be comparable to an open cable tie, zip tie, or similar ratcheting type fastener.

For example, to transition from an open position as shown in FIG. 2a to the first closed position shown in FIG. 2b, beveled edge 114 may contact first tooth 124. A downward force may be exerted on horizontal arm 112, such that beveled edge 114 may slide down the downward slope of first tooth 124 and engage a backside of first tooth 124. For example, a surgeon or other medical professional may exert a downward force by pressing horizontal arm down with their finger or thumb. In this position, the backside of the first tooth 124 may act against a resilient force, for example a torque or lever force, exerted on horizontal arm 112 proximate second end 108, which may stop clamp 102 from returning to the open position. Further, during the transition from the open position to the first closed position, first protrusion 120 and second protrusion 122 may converge, which may increase a compressing force, exerted on either side of surgical tubing 104, which may at least partially restrict a flow of fluid through surgical tubing 104.

FIG. 2c may show clamp 102 in a second closed position. For example, in the second closed position, beveled edge 114 of horizontal arm 112 may engage a last tooth 126 of said plurality of teeth 118 of vertical arm 116. As noted above, beveled edge 114 may act, for example as a pawl, and plurality of teeth 118 may act, for example, as a rack, as would be understood by a person of ordinary skill in the art. Thus, in one exemplary embodiment, clamp 102 may transition from the first closed position as shown in FIG. 2b to the second closed position shown in FIG. 2c by exerting an additional downward force on horizontal arm 112.

For example, an additional downward force may be applied on horizontal arm such that beveled edge 114 may disengage the backside of first tooth 124. Beveled edge 114 may slide down the downward slope of the second tooth to engage the backside of the second tooth. Additional downward pressure may be applied to slide or ratchet beveled edge 114 down each successive tooth of the plurality of teeth 118 until engaging backside of last tooth 126 as would be understood by a person of ordinary skill in the art. In this position, the backside of the last tooth 126 may act against a resilient force, for example a torque or lever force, exerted on horizontal arm 112 proximate second end 108, which may stop clamp 102 from returning to the open position or any other closed position. Further, during the transition from the open position to the first closed position and subsequently to the second closed position, first protrusion 120 and second protrusion 122 may converge with each successive step. Accordingly, an increased compressing force may be exerted on either side of surgical tubing 104, which may at least partially restrict a flow through surgical tubing 104.

Referring generally to FIGS. 2a-c, to transition clamp 102 from a closed position to the open position, a user may exert a force on vertical arm 116 in the direction of first end 106. Beveled edge 114 may disengage from the backside of one of the plurality of teeth 118. A resilient force, for example a torque or lever force, may act on horizontal arm 112 proximate second end 108, to return clamp 102 to the open position.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A surgical tube clamp, comprising:
a hollow body with a first end a second end;
a bottom surface between the first end and the second end;
a vertical arm extending from the first end, the vertical arm having an inner face with a plurality of teeth;
a horizontal arm extending from the second end toward the first end, the horizontal arm having a beveled edge proximate the inner face;
a first protrusion on the bottom surface extending within the hollow body; and
a second protrusion on the horizontal arm extending within the hollow body;
wherein a first force may be applied on the body such that the horizontal arm and the vertical arm converge to engage the beveled edge with a backside of one of the plurality of teeth to transition the clamp from an open position to a first closed position.

2. The surgical tube clamp of claim 1, wherein an additional force may be applied to the horizontal arm to engage a backside of another of the plurality of teeth to transition the clamp from the first closed position to at least one second closed position.

3. The surgical tube clamp of claim 1, wherein a second force may be applied on the body to disengage the beveled edge from the backside of one of the plurality of teeth to transition the clamp from the first closed position to the open position.

4. The surgical tube clamp of claim 1, wherein a distance between the first protrusion and the second protrusion is greater in the open position than in the first closed position.

5. The surgical tube clamp of claim 2, wherein a distance between the first protrusion and the second protrusion is greater in the first closed position than in in the at least one second closed position.

6. The surgical tube clamp of claim 1, wherein the plurality of teeth are downward sloping.

7. The surgical tube clamp of claim 1, wherein beveled edge acts as a pawl and the plurality of teeth act as a rack.

8. The surgical tube clamp of claim 1, wherein the body is at least one of an oval, oblong or rectangular shape.

9. The surgical tube clamp of claim 1, wherein the first protrusion and the second protrusion are rounded.

10. A system for silent surgical tubing, comprising:
a surgical clamp with a hollow body having a first end a second end;
a first aperture in the first end;
a second aperture in the second end;
a bottom surface between the first end and the second end;
a vertical arm extending from the first end, the vertical arm having an inner face with a plurality of teeth;
a horizontal arm extending from the second end toward the first end, the horizontal arm having a beveled edge proximate the inner face;
a first protrusion on the bottom surface extending within the hollow body; and
a second protrusion on the horizontal arm extending within the hollow body;
a surgical tube received through the first aperture and the second aperture, and operable to draw a fluid in a direction from the first end to the second end;
wherein a first force may be applied on the body such that the horizontal arm and the vertical arm converge to engage the beveled edge with a backside of one of the plurality of teeth to transition the clamp from an open position to a first closed position.

11. The system of claim 10, wherein the first protrusion and the second protrusion converge when in the first closed position to apply a compressive force on the surgical tube to at least partially restrict a fluid flow through the surgical tube.

12. The system of claim 11, wherein a second force may be applied to the horizontal arm to engage a backside of another of the plurality of teeth to transition the clamp from the first closed position to at least one second closed position, wherein in the at least one second closed position, the first protrusion and the second protrusion further converge to apply an increased compressive force on the surgical tube to further restrict the fluid flow through the surgical tube.

13. The system of claim 10, wherein the clamp may slide along a length of the surgical tube.

14. The system of claim 12, wherein the beveled edge acts as a pawl and the plurality of teeth act as a rack to ratchet the clamp from the first closed position to the at least one second closed position.

15. The system of claim 10, wherein a second force may be applied on the body to disengage the beveled edge from the backside of one of the plurality of teeth to transition the clamp from the first closed position to the open position.

16. The system of claim 10, further comprising:
at least one end clip on the surgical tubing to impede the movement of the surgical clamp along the length of the surgical tubing.
